(19) United States
(12) Patent Application Publication
(10) Pub. No.: US 2019/0046006 A1
(43) Pub. Date: Feb. 14, 2019

(54) DRY SANITIZATION APPARATUS COMPRISING COPPER

(71) Applicant: Shirin Ahadpour Dorin, Escondido, CA (US)

(72) Inventor: Shirin Ahadpour Dorin, Escondido, CA (US)

(21) Appl. No.: 16/100,061

(22) Filed: Aug. 9, 2018

Related U.S. Application Data
(60) Provisional application No. 62/543,717, filed on Aug. 10, 2017.

(51) Int. Cl. A47L 23/26; A61L 2/238

(52) U.S. Cl. CPC A47L 23/266; A61L 2202/11; A61L 2/238

(57) ABSTRACT

A dry sanitization apparatus or mat comprising copper is disclosed. Systems comprising the mat and methods of use are also disclosed.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the benefit of U.S. Provisional Patent Application No. 62/543,717, filed Aug. 10, 2017, entitled “Copperhead Mat,” the content of which is hereby incorporated by reference in its entirety.

### BACKGROUND

It is important to disinfect disease sensitive areas, such as hospitals, homes, offices, schools, gyms, nursing homes, daycare centers and assistant living facilities, in order to prevent infection and the spread of illness. Environments such as these not only house individuals that are immunocompromised and therefore are most at risk to diseases, but also are breading grounds for spreading infections amongst the general public.

Traditionally, antimicrobials and harsh cleaning methods have been used to disinfect environments. However, increasing antimicrobial resistance and the fact that many infectious diseases can survive harsh cleaning methods are of increasing concern. Furthermore, there is a long standing problem of microbes being brought into disease sensitive areas on the soles of shoes and the bottom of feet. Solvent based systems used to sanitize feet and/or shoes are impractical in many disease sensitive settings, such as hospitals, homes, offices, schools, gyms, nursing homes, daycare centers and assistant living facilities, because of the need to keep these settings dry as well as the required cleaning and refilling of the solvent based systems. As such, novel sanitization methods and systems that can be practically utilized in disease sensitive settings are needed.

### SUMMARY

In one aspect, a dry sanitization mat is provided. The dry sanitation mat includes a copper element comprising copper, wherein the copper element is biocidal. The mat further includes a frame, wherein the copper element is housed in the frame. The mat further includes wherein the mat is solvent free.

In some embodiments of the dry sanitation mat, the copper element comprises elemental copper. In some embodiments, the copper element is at least one sheet of elemental copper. In some embodiments, the elemental copper is hard, nonporous copper.

In some embodiments, the frame comprises a frame slot configured to allow insertion and/or removal of the copper element. In some embodiments, the mat further comprises legs attached to the frame. In some embodiments, the frame comprises rubber.

In some embodiments, the copper element is bactericidal. In some embodiments, the copper element is bactericidal to at least one of Escherichia coli, Methicillin-Resistant Staphylococcus aureus (MRSA), Staphylococcus aureus, Vancomycin-Resistant Enterococcus faecalis (VRE), Enterobacter aerogenes and Pseudomonas aeruginosa. In some embodiments, the copper element is at least about 50% bactericidal. In some embodiments, the mat is at least about 80% bactericidal. In some embodiments, the copper element is at least about 90% bactericidal. In some embodiments, the copper element is at least about 95% bactericidal. In some embodiments, the copper element is at least about 99% bactericidal.

In some embodiments, the mat further comprises a support member.

In another aspect a sanitization system comprising an entryway and at least one dry sanitation mat is provided. The system includes wherein the at least one mat is positioned directly in front of, directly behind or within the entryway.

In some embodiments of the system, the entryway is located at the entrance of or within a disease sensitive setting. In some embodiments, the disease sensitive setting is a hospital, home, office, school, gym, automobile, nursing home, daycare center or assistant living facility. In some embodiments, the at least one mat is affixed directly in front of, directly behind or within the entryway. In some embodiments, the mat is positioned on the floor or ground.

In another aspect a dry sanitization floor mat is provided. The dry sanitation mat includes a copper element consisting of a hard, nonporous elemental copper sheet, wherein the copper element is bactericidal, and wherein the copper element comprises an outer edge region. The mat further includes a frame, wherein the copper element is housed in the frame, and wherein the frame comprises a frame side member and a frame extension. The mat further includes the frame side member having an outer wall and an inner wall. The mat further includes a support member positioned inside the inner wall of the frame side member and adjacent the copper element. The mat further includes wherein the mat is solvent free. The mat further includes wherein the frame extension is positioned to engage the outer edge region of the copper element so as to help secure the copper element within the frame.

In some embodiments of the dry sanitation mat, the frame comprises a slot configured to removably receive the copper element. In some embodiments, the support member is integrally connected to the frame side member. In some embodiments, the copper element is at least about 99% bactericidal.

### BRIEF DESCRIPTION OF THE DRAWINGS

FIG. 1 illustrates a perspective view of one embodiment of the present disclosure.

FIG. 2 illustrates a side view of one embodiment of the present disclosure.

FIG. 3 illustrates a top view of one embodiment of the present disclosure.

FIG. 4 illustrates a bottom view of one embodiment of the present disclosure.

FIG. 5 illustrates a cross-sectional view of one embodiment of the present disclosure.

FIG. 6 illustrates a side view of a second embodiment of the present disclosure comprising a slot.

### DETAILED DESCRIPTION

In order to prevent pathogens from entering or being passed through disease sensitive settings, such as hospitals, homes, offices, schools, gyms, nursing homes, daycare centers, assistant living facilities and other similar spaces, and to overcome the problems with existing sanitization systems, the present disclosure describes a dry sanitization apparatus or mat comprising copper. In some instances, the apparatus may be positioned in front of, behind or within an entry way. For example, the apparatus or mat may be used as a doormat for the front door of a home or apartment. In another example, the mat may be used as a foot and/or shoe disinfecting mat before an individual enters a room within a hospital. The term “dry” with regard to the apparatus refers to an apparatus that is absent of solutions and/or solvent in its normal mode of operation, and it should be appreciated that trace amounts of solvent within the apparatus and incidental solutions, such as water, over the apparatus are still to be considered “dry” for the purposes of this disclosure.

Various beneficial characteristics of embodiments of the mat comprising copper are disclosed herein. In some embodiments, the copper provides biocidal properties. In some embodiments, the copper provides antimicrobial properties. In some embodiments, the copper provides bactericidal properties. In some embodiments, the copper is bactericidal to Staphylococcus aureus. In some embodiments, the copper is bactericidal to Escherichia coli. In some embodiments, the copper is bactericidal to Pseudomonas aeruginosa. In some embodiments, the copper is bactericidal to Enterococcus faecalis. In some embodiments, the copper is bactericidal to Enterobacter aerogenes. In some embodiments, the copper is bactericidal to resistant bacteria, such as, for example, Methicillin-Resistant Staphylococcus aureus (MRSA) and/or Vancomycin-Resistant Enterococcus faecalis (VRE). In some embodiments, the copper provides fungicidal properties. In some embodiments, the copper prevents the growth of fungi and fungal spores. In some embodiments, the copper degrades the DNA of microorganisms when in or after contact. In some embodiments, the copper interferes with cellular respiration and/or metabolic functions of microorganisms when in or after contact. In some embodiments, the copper provides virucidal properties. In some embodiments, the copper is virucidal to norovirus. In some embodiments, the copper is virucidal to coronavirus. In some embodiments, the copper is virucidal to influenza A virus. In some embodiments, the copper is virucidal to adenovirus.

In some embodiments, the dry sanitization mat comprises a copper element. In some embodiments, the copper element comprises elemental copper. In some embodiments, the copper element is at least one sheet of elemental copper. In some embodiments, the copper element is a sheet of elemental copper. In some embodiments, the elemental copper is hard, nonporous copper. In some embodiments, the elemental copper sheet is a CuVerro® copper sheet. In some embodiments, the copper element is secured to the mat by glue. In some embodiments, the copper element is secured to the mat by fasteners. In some embodiments, the copper element is secured to the mat by the frame, such as being gripped or held in place by the frame.

In some embodiments, the copper element is bactericidal. In this context, “bactericidal,” when referring to an element, is an element which has properties that meet standards set by an appropriate regulatory body (e.g., by the U.S. Environmental Protection Agency). A bactericidal copper element having such properties is considered to meet the standards regardless of whether the regulatory body has provided official notification to that effect. In some embodiments, bactericidal refers to the copper element is at least 50% (or about 50%), at least 60% (or about 60%), at least 70% (or about 70%), at least 80% (or about 80%), at least 90% (or about 90%), at least 95% (or about 95%), at least 99% (or about 99%), at least 99.5% (or about 99.5%), at least 99.9% (or about 99.9%) or at least 99.99% (or about 99.99%) bactericidal, or any range between any of these values. In some embodiments, the degree (e.g. percent) of bactericidal properties may be accomplished by the copper element in less than 1 second (or about less than 1 second), 1 second (or about 1 second), 30 seconds (or about 30 seconds), 1 minute (or about 1 minute), 30 minutes (or about 30 minutes), 1 hour (or about 1 hour), 2 hours (or about 2 hours), 3 hours (or about 3 hours) or 4 hours (or about 4 hours), or any range between any of these values.

In some embodiments, the dry sanitization mat comprises a frame. In some embodiments, the copper element is housed in the frame. In some embodiments, the frame comprises a frame slot. In some embodiments, the frame slot is configured to allow insertion and/or removal of the copper element. In some embodiments, the copper element is fixedly attached to the frame. In some embodiments, the frame comprises an exterior frame portion and an interior frame portion. In some embodiments, the frame comprises rubber.

In some embodiments, the dry sanitization mat comprises a support member. In some embodiments, the support member is positioned within the frame. In some embodiments, the support member is positioned exterior to the frame. In some embodiments, the support member is integral with the frame such that the frame is a single monolithic structure. In some embodiments, the support member is a filler material, such as polyurethane and/or polyurea. In some embodiments the filler material is a Chemline® polyurethane and/or polyurea filler, such as Chemline® TDEA80.5-21-M20.

In some embodiments, the dry sanitization mat comprises legs. In some embodiments, the legs are attached to the frame.

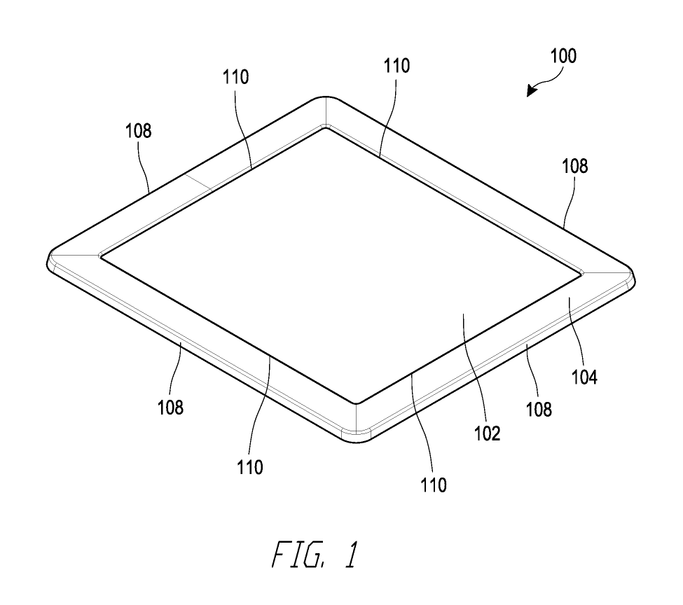

FIGS. 1-5 illustrate one embodiment of a mat 100 comprising copper. FIG. 1 illustrates a perspective view of a mat 100 comprising a copper element 102 disposed or housed within a frame 104. The frame 104 is illustrated as a square frame, wherein the frame 104 comprises a plurality of outer walls 108 on the exterior side surfaces of the mat 100. Frame 104 is also illustrated with a plurality of frame lips 110, wherein the frame lips are curved and positioned over the copper element 102 such that the copper element 102 is recessed within the mat 100.

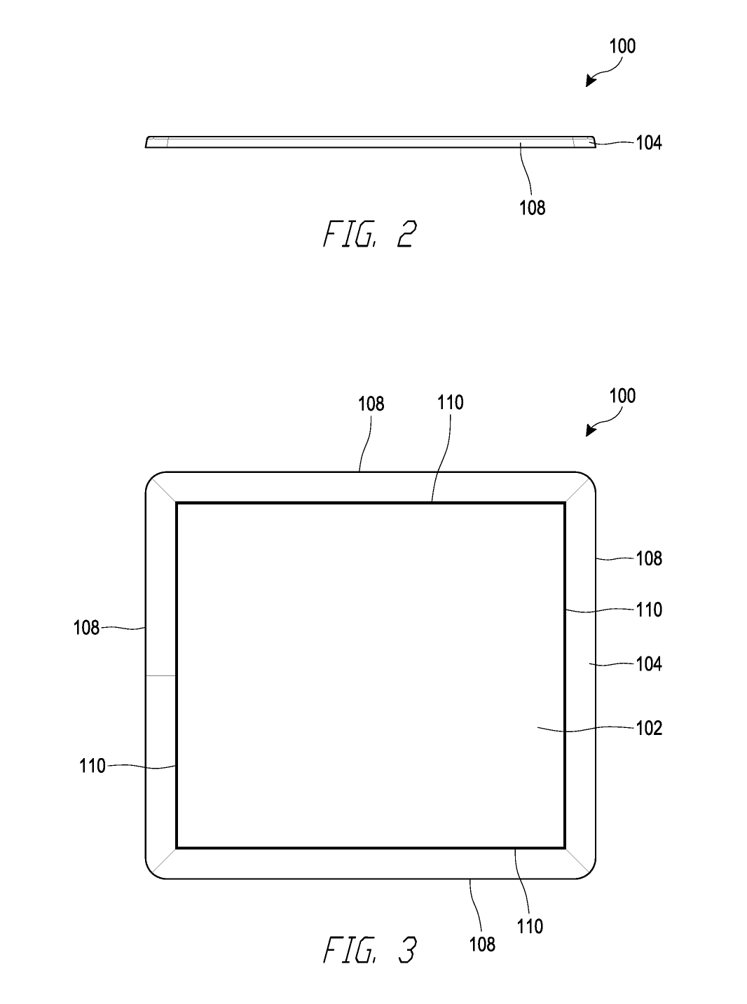

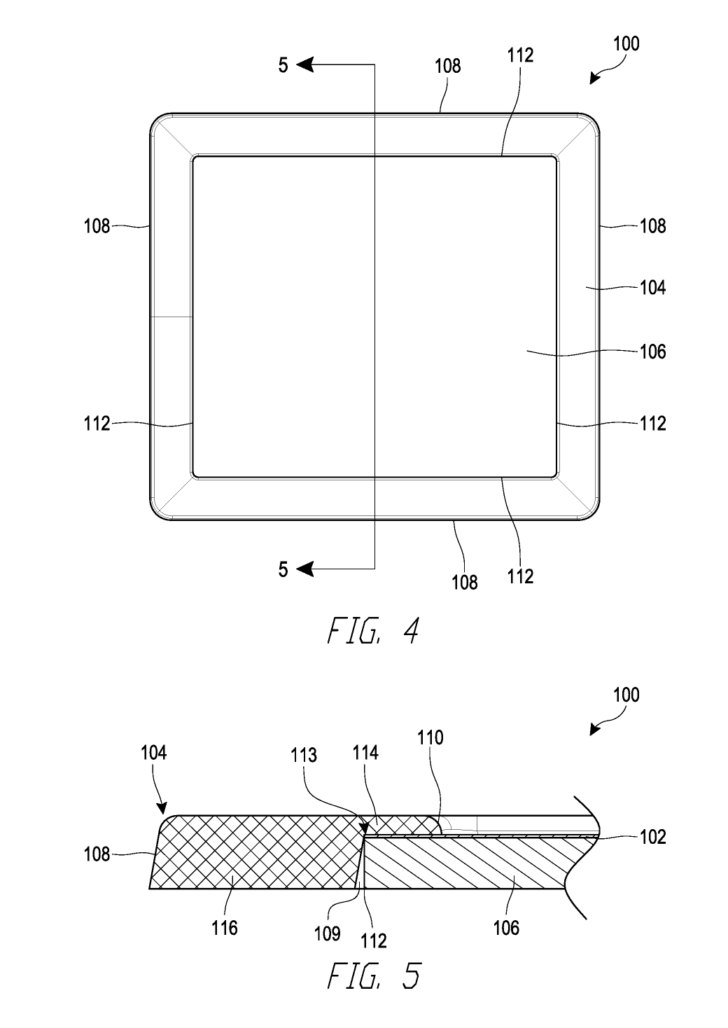

FIG. 2 illustrates a side view of the mat 100 of FIG. 1, wherein the frame 104 is illustrated with an outer wall 108. FIG. 3 illustrates a top view of the mat 100 with copper element 102, frame 104 having outer walls 108 and frame lips 110. FIG. 4 illustrates a bottom view of the mat 100 with frame 104, frame outer walls 108, inner walls 109 and further comprising a support member 106 positioned or housed within the frame and a plurality of support member edges 112. Support member edges 112 are positioned adjacent frame inner walls 109.

FIG. 5 illustrates a cross-sectional view of the mat 100, as indicated in FIG. 4. The mat 100 comprises frame 104, support member 106 shown where frame 104 is cut away for illustrative purposes, and copper element 102 housed within the frame 106 and positioned adjacent a support member 106. Frame 104 comprises frame side member 116 and extension 114. Frame side member 116 includes an outer wall 108 on the outer rim of the frame 104 and an inner wall 109 on the opposite end of the frame side member 116. Inner wall 109 of frame side member 116 lies adjacent support member edge 112. Frame extension 114 comprises a frame lip 110. Lip 110 is shown with curved or beveled edges. Copper element 102 comprises outer edge region 113, which contacts inner wall 109 of the frame support member 116 and extension 114. Extension 114 extends beyond inner wall 109 and support member edge 112 and is positioned over the outer edge region 113 of copper element 102 so as to secure copper element 102 in position within frame 104.

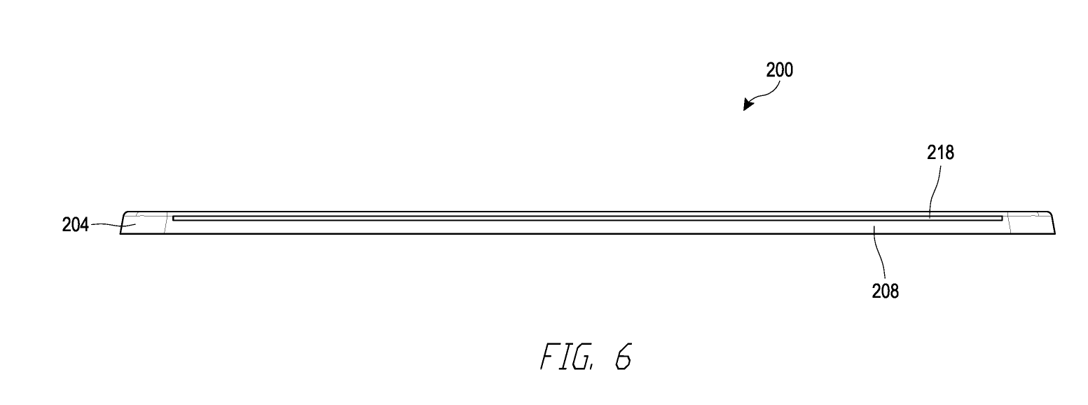

FIG. 6 illustrates a side view of a second embodiment of a mat 200 similar to the mat illustrated in FIGS. 1-5. Mat 200 comprises a frame 204 having frame edge 208, wherein frame edge 208 comprises slot 218 positioned so as to allow a copper element (not shown) to be removable.

### Sanitization System

In some embodiments, a sanitization system comprising an entryway and the dry sanitization apparatus or mat is described. Various benefits of such a sanitization system include preventing, reducing or minimizing exposure and/or transfer of dangerous pathogens. Furthermore, such benefits may be accomplished without the use of solutions, solvents or particles, the use of which may not be practically utilized in many environments. It should be appreciated that these benefits may also be realized with the mat alone, or in combination with a system.

In some embodiments, a sanitization system comprising an entryway and at least one dry sanitization mat. In some embodiments, the mat is positioned directly in front of, directly behind or within the entryway. In some embodiments, the mat is positioned on the floor or ground. In some embodiments, the mat is affixed directly in front of, directly behind or within the entryway. In some embodiments, the entryway is located at the entrance of or within a disease sensitive setting. The sanitization system may be integrated into an automatic door activation system, such as an activation floor mat. In some embodiments, the disease sensitive setting is a hospital, home, office, school, gym, automobile, nursing home, daycare center or assistant living facility. In some embodiments, the entryway is a doorway and/or a doorframe.

### Method of Use

The apparatuses or mats and systems described herein may be used in various methods.

In some embodiments, the method comprises contacting an extremity with the mat. In some embodiments, the method comprises contacting a shoe or a foot with the mat. In some embodiment, contacting the mat or copper element produces antibacterial effects. In some embodiments, contacting an extremity with the mat or copper element is at least 50% (or about 50%), at least 60% (or about 60%), at least 70% (or about 70%), at least 80% (or about 80%), at least 90% (or about 90%), at least 95% (or about 95%), at least 99% (or about 99%), at least 99.5% (or about 99.5%), at least 99.9% (or about 99.9%) or at least 99.99% (or about 99.99%) bactericidal, or any range between any of these values. In some embodiments, bactericidal properties are achieved by contacting an extremity with the mat or copper element for less than 1 second (or about less than 1 second), 1 second (or about 1 second), 30 seconds (or about 30 seconds), 1 minute (or about 1 minute), 30 minutes (or about 30 minutes), 1 hour (or about 1 hour), 2 hours (or about 2 hours), 3 hours (or about 3 hours) or 4 hours (or about 4 hours), or any range between any of these values. In some embodiments, the extremity is a shoe and/or a foot. In some embodiments, the extremity is a hand.

### Examples

Provided herein are experimental examples of a copper element producing antibacterial properties. The experimental examples demonstrate that a hard, non-porous copper containing surface unexpectedly shows antimicrobial properties against a number of microbes commonly spread on floors by shoes and/or feet.

The antimicrobial properties of a hard, non-porous copper containing surface was evaluated following the exposure time of 1 hour at 22.4° C. and 39.15% relative humidity (RH) against organisms Staphylococcus aureus 6538, Escherichia coli 25922, and Pseudomonas aeruginosa 15442. The test methodology was adapted from the U.S. Environmental Protection Agency's (EPA's), “Protocol for the Evaluation of Bactericidal Activity of Hard, Non-porous Copper Containing Surface Products (Jan. 29, 2016),” herein incorporated by reference in its entirety.

S. aureus, E. coli and P. aeruginosa: bacterial cultures were initiated 22±2 hours prior to testing by inoculating 10 ml of tryptic soy broth (TSB) with one colony of the respective bacteria. On the day of testing, bacterial cells were pelleted by centrifugation (4,000×g for 10 minutes), and washed twice using 0.01M phosphate buffered saline (PBS) with successive rounds of centrifugation. On the day of testing, the carrier inoculum culture was diluted to achieve target inocula of 5×105 organisms per 0.010 ml by measuring the optical density (OD) of the culture at a wavelength of 600 nm. A target absorbance between 0.115 and 0.120 was sought for a concentration of 1×108 cells/ml. For both the test exposure challenge and the neutralization validation, organisms were diluted to obtain an absorbance between 0.115 and 0.120. The cells were further diluted to achieve 5×105 cells/0.010 ml or 5×105 cells/carrier. Inoculum cultures were amended with a tri-partite soil solution composed of 5% Bovine Serum Albumin, 7% Yeast Extract, and 20% Mucin.

Carriers were mounted in sterile Petri dishes and inoculated with 0.010 ml of the test cultures in replicates of two (2) according to the following: a) two (2) Time Zero Stainless Steel Control Carriers (to be harvested for enumeration immediately); b) two (2) Stainless Steel carriers per organism held at 22.4° C. and 39.15% relative humidity for one (1) hour; c) two (2) Copper carriers per organism held at 22.4° C. and 39.15% relative humidity for one (1) hour; and d) two (2) carriers (One Copper, One Stainless Steel) per organism to validate the neutralization step of the experiment were held at 22.4° C. and 39.15% relative humidity for one (1) hour. These were inoculated with PBS which contained only the tri-partite soil load.

Inoculated test carriers were held in a sterile environment (biosafety cabinet) for one (1) hour. Duplicate inoculated stainless steel control carriers were harvested immediately upon drying into 20 ml of Dey/Engley (D/E) Broth in stomacher bags, and served as Time Zero Controls. Following a 60 second stomaching, the detached organisms were diluted 10-fold. Bacterial cultures were plated onto tryptic soy agar (TSA) and incubated for 48 hours at 37° C. for enumeration. After the designated exposure time, the test carriers were harvested and plated in the same manner as the Time Zero Stainless Steel Control carriers.

For neutralization validation, D/E broth from the stomaching of each carrier type (blank copper carrier and blank stainless steel carrier) was saved and test organisms serially diluted in each series of D/E to achieve a final count of 5×101 colonies. Comparable growth (less than 50% difference) between the copper carrier D/E solution and stainless steel control D/E solution indicates proper neutralization. All platings were incubated for ˜48 hours at 37° C. Bacterial colony-forming units (CFUs) were then enumerated, and the reductions calculated.

Tables 1-3 show the results of the antimicrobial tests.

TABLE 1
aureus 6538 Data*^{,}**

| | | Bacterial | | | |
|---|---|---|---|---|---|
| | | Titer | Geomean | | |
| Carrier | | (CFU per | Bacterial | Log10 | Percent |
| Designation | Contact Time | Carrier) | Titer | Reduction | Reduction |
| Stainless Steel | Time Zero | 3.28E+05 | 3.62E+05 | N/A | N/A |
| Control | | 3.99E+05 | | | |
| Stainless Steel | 1 Hour | 2.55E+05 | 2.31E+05 | 0.19 | 36% |
| Control | | 2.10E+05 | | | |
| Copper Test | 1 Hour | ≤2.00E+01 | ≤2.00E+01 | ≥4.06 | ≥99.991% |
| | | ≤2.00E+01 | | | |

*Log10 and Percent Reduction for Copper Test calculated relative to 1-Hour Stainless Steel Control
**“≤”No growth observed; therefore, bacterial levels below the limit of detection

TABLE 2
E. coli 25922 Data*^{,}**

| | | Bacterial | | | |
|---|---|---|---|---|---|
| Carrier | | Titer | Geomean | | |
| Desig- | Contact | (CFU per | Bacterial | Log10 | Percent |
| nation | Time | Carrier) | Titer | Reduction | Reduction |
| Stainless | Time | 3.67E+05 | 3.78E+05 | N/A | N/A |
| Steel | Zero | | | | |
| Control | | 3.90E+05 | | | |
| Stainless | 1 Hour | 1.37E+04 | 1.46E+04 | 1.41 | 96% |
| Steel | | | | | |
| Control | | 1.56E+04 | | | |
| Copper | 1 Hour | ≤2.00E+01 | ≤2.00E+01 | ≥2.86 | ≥99.8% |
| Test | | ≤2.00E+01 | | | |

*Log10 and Percent Reduction for Copper Test calculated relative to 1-Hour Stainless Steel Control
**“≤”No growth observed; therefore, bacterial levels below the limit of detection

TABLE 3
P. aeruginosa 15442 Data*^{,}**

| | | Bacterial | | | |
|---|---|---|---|---|---|
| Carrier | | Titer | Geomean | | |
| Desig- | Contact | (CFU per | Bacterial | Log10 | Percent |
| nation | Time | Carrier) | Titer | Reduction | Reduction |
| Stainless | Time | 5.62E+05 | 4.84E+05 | N/A | N/A |
| Steel | Zero | | | | |
| Control | | 4.17E+05 | | | |
| Stainless | 1 Hour | 1.96E+04 | 1.60E+04 | 0.48 | 67% |
| Steel | | | | | |
| Control | | 1.31E+04 | | | |
| Copper | 1 Hour | ≤2.00E+01 | ≤2.00E+01 | ≥3.90 | ≥99.98% |
| Test | | ≤2.00E+01 | | | |

*Log10 and Percent Reduction for Copper Test calculated relative to 1-Hour Stainless Steel Control
**“≤”No growth observed; therefore, bacterial levels below the limit of detection

Tables 1-3 demonstrate that for both Gram-positive (S. aureus) and Gram-negative (E. coli and P. aeruginosa) microorganisms dried onto hard, nonporous copper surfaces, the copper surfaces exhibited a greater than 4-log 10 (>99.99%) reduction for all organisms within 1 hour (compared to the stainless steel timed controls).

It will be apparent to those skilled in the art that various modifications and variations can be made in the present invention without departing from the scope or spirit of the invention. Thus, it is intended that the present invention cover the modifications and variations of this invention provided that they come within the scope of the appended claims or their equivalents.

What is claimed is:

1. A dry sanitization mat comprising:
a copper element comprising copper, wherein the copper element is biocidal; and
a frame, wherein the copper element is housed in the frame;
wherein the mat is solvent free.

2. The mat of claim 1, wherein the copper element comprises elemental copper.

3. The mat of claim 2, wherein the copper element is at least one sheet of elemental copper.

4. The mat of claim 3, wherein the elemental copper is hard, nonporous copper.

5. The mat of claim 1, wherein the frame comprises a frame slot configured to allow insertion and/or removal of the copper element.

6. The mat of claim 1, further comprising legs attached to the frame.

7. The mat of claim 1, wherein the frame comprises rubber.

8. The mat of claim 1, wherein the copper element is bactericidal.

9. The mat of claim 8, wherein the copper element is bactericidal to at least one of Escherichia coli, Methicillin-Resistant Staphylococcus aureus (MRSA), Staphylococcus aureus, Vancomycin-Resistant Enterococcus faecalis (VRE), Enterobacter aerogenes and Pseudomonas aeruginosa.

10. The mat of claim 8, wherein the copper element is at least about 50% bactericidal.

11. The mat of claim 10, wherein the mat is at least about 80% bactericidal.

12. The mat of claim 11, wherein the copper element is at least about 90% bactericidal.

13. The mat of claim 12, wherein the copper element is at least about 95% bactericidal.

14. The mat of claim 13, wherein the copper element is at least about 99% bactericidal.

15. The mat of claim 1, further comprising a support member.

16. A sanitization system comprising an entryway and at least one mat of claim 1, wherein the at least one mat is positioned directly in front of, directly behind or within the entryway.

17. The system of claim 16, wherein the entryway is located at the entrance of or within a disease sensitive setting.

18. The sanitization system of claim 17, wherein the disease sensitive setting is a hospital, home, office, school, gym, automobile, nursing home, daycare center or assistant living facility.

19. The system of claim 16, wherein the at least one mat is affixed directly in front of, directly behind or within the entryway.

20. The system of claim 16, wherein the mat is positioned on the floor or ground.

21. A dry sanitization floor mat comprising:
a copper element consisting of a hard, nonporous elemental copper sheet, wherein the copper element is bactericidal, and wherein the copper element comprises an outer edge region;
a frame, wherein the copper element is housed in the frame, and wherein the frame comprises a frame side member and a frame extension;
the frame side member having an outer wall and an inner wall; and
a support member positioned inside the inner wall of the frame side member and adjacent the copper element;
wherein the mat is solvent free; and
wherein the frame extension is positioned to engage the outer edge region of the copper element so as to help secure the copper element within the frame.

22. The mat of claim 21, wherein the frame comprises a slot configured to removably receive the copper element.

23. The mat of claim 21, wherein the support member is integrally connected to the frame side member.

24. The mat of claim 21, wherein the copper element is at least about 99% bactericidal.
